(19) United States
(12) Patent Application Publication
(10) Pub. No.: US 2016/0237378 A1
(43) Pub. Date: Aug. 18, 2016

(54) A FILTER MEDIUM FOR THE FILTRATION OF OLIVE OIL, IN PARTICULAR FOR THE FILTRATION OF EXTRA VIRGIN OLIVE OIL, AND THE FILTRATION PROCESS OF THE OLIVE OIL, IN PARTICULAR OF THE FILTRATION OF THE EXTRA VIRGIN OLIVE OIL

(71) Applicant: Massimo EPIFANI, Foligno (PG) (IT)

(72) Inventor: Massimo EPIFANI, Foligno (PG) (IT)

(21) Appl. No.: 15/024,927

(22) PCT Filed: Sep. 26, 2014
(86) PCT No.: PCT/IB2014/001930

§ 371 Date: Mar. 25, 2016

(30) Foreign Application Priority Data
Sep. 27, 2013 (IT) AR2013A000036

(51) Int. Cl. C11B 3/00; C10L 5/48; C10L 5/36; A23K 20/158; A61K 8/73; A61Q 19/00; C05F 5/00; B01D 39/04; C10L 5/44; A61K 8/97

(52) U.S. Cl. CPC C11B 3/008; C10L 5/44; C10L 5/48; C10L 5/363; A61K 8/97; A61K 8/731; A61Q 19/00; C05F 5/002; B01D 39/04; A23K 20/158; C10L 2200/0469; A61K 2800/10

(57) ABSTRACT

A filter medium for the filtration of olive oil, in particular for the filtration of extra virgin olive oil, and filtration process of the olive oil, in particular of the filtration of extra virgin olive oil, which has the peculiarity of being composed of at least 95% by weight, on the dry material, of cellulose.

### TECHNICAL FIELD

The present invention relates to a filter medium for the filtration of olive oil, in particular for the filtration of extra virgin olive oil, and to the filtration process of the olive oil, in particular the filtration of extra virgin olive oil.

### BACKGROUND ART

The production of oil from the drupes of the olive is a process not exclusively industrial that is divided roughly in a continuous cycle in which the fruit is ground (crushing and/or milling) with subsequent separation (centrifugal) of the oil fraction from the water.

The filtration is a physical-mechanical operation with which a moving liquid, under the action of a pressure gradient, is separated from the solid particles dispersed in it, for the effect of their retention by a porous adjuvant through which the liquid is made to pass.

The retention of the particles contained in the liquid process is implemented by adjuvants of the filter media. These filtration adjuvants, which cling on the filtering mesh (or septum) shall also ensure a solid connection with the support of filtration and a subsequent detachment of the deposit from such support at the end of the operation.

The most common adjuvants, used in the olive oil industry for the filtration of olive oil are fossil flour.

The fossil flour is a fine powder (microscopic weave of fibers) obtained from the processing of fossil shells of microscopic algae of the class of diatoms. For this reason it is also called diatomite.

At the base of its chemical composition, there is a high percentage of calcium carbonate and silicon dioxide.

This type of adjuvant is widely used because it has special filtering capacity. It is distinguished by its variable range of porosity, for the huge development of its surface as well as to its low density.

At the current state of the art, it is known to add adjuvants to the fossil flour, such as parts of cellulose of plant origin, normally in an amount included between 20 and 30% by weight.

This cellulose is obtained from the raw wood through a series of physical and chemical treatments and is used primarily to form the first filter layer placed against the meshes of the filter with the aim to prevent the cohesion of fossil flour.

A first problem related to the use of fossil flour, is given by the fact that the raw material supply is being depleted and requires to be reconstituted in a geological process.

Besides that, the filtration of olive oil with this type of filter, produces at the end of the cycle exhausted filtration adjuvants impregnated of oil which cannot be sent to a landfill but must be disposed of in a suitable manner to avoid damage to the environment, such as causing the turbidity of the water which damages the biotic components.

The exhausted fossil flour then are taken to disposal waste water sites and to the treatment plants of the same, with substantial costs borne by the company.

Another drawback due to the use of fossil flour is given by the fact that they contain crystalline silica, a harmful substance with irreversible effects for humans since it causes silicosis, a serious lung disease. Even the IARC (International Agency for Research of Cancer) has classified crystalline silica as carcinogenic by inserting it between the 111 elements of group 1, which are harmful to humans.

For this reason, fossil flour have in their original packaging the St. Andrew's cross, the symbol of acute toxicity in the labels in accordance with the EU regulation and standardized with respect to the parameters GHS (Globally Harmonized System of Classification and Labelling of Chemicals) set by the United Nations in 2002, and therefore must be handled with extreme care and attention in order to avoid any risk of inhalation by operators.

### DISCLOSURE OF THE INVENTION

The aim of the invention is to eliminate the drawbacks noted above in known types of filter media for filtration of olive oil, which allows to filter the olive oil, without exposing operators to serious risks of disease.

Within the aforementioned aim, a purpose of the present invention is to obtain a byproduct of filtration, which is easily disposable and, in the case, can be recycled in a manner compatible with respect to the environment.

Another purpose of the filter medium according to the invention, is to use a renewable material in such a way that it is practically inexhaustible.

Another purpose of the present invention is to allow a filtration of olive oil that is safer than the traditional one and which allows to reuse the exhausted adjuvants in an economically advantageous and easily manageable manner.

Another object of the invention is to become an alternative means to the use of diatomite as adjuvant, given that the diatoms are running out.

Another object of the invention is to provide a filter medium for the filtration of olive oil, in particular for the filtration of extra virgin olive oil, with means readily available on the market as well as using materials in common use, so that the device is even more competitive.

This aim, these objects and others which will become apparent hereinafter, are achieved by a filter medium for the filtration of olive oil, in particular for the filtration of extra virgin olive oil, according to the invention, characterized in that consists of at least 95% by weight, on the dry material, of cellulose.

Further characteristics and advantages of the invention will become apparent from the description of a preferred embodiment, but not exclusive, of the filter medium for the filtration of olive oil, in particular for the filtration of extra virgin olive oil, and use of the exhausted panel of filtration of the olive oil, in particular the filtration of extra virgin olive oil.

### MODES FOR CARRYING OUT THE INVENTION

The filter medium according to the invention, has the fundamental characteristic of being constituted by at least 95% by weight, on dry material, of cellulose.

Advantageously, the filter medium is made only of cellulose, in particular of cellulose fiber in use in the food industry.

Preferably, the filter medium has a moisture included between 5 and 7%, so as to avoid the presence of moisture in the bottle which jeopardizes the quality of oil in storage.

Preferably, in this regard, the filter medium has a porosity filter included between 2 and 13 Darcy.

The filtration process of the olive oil, in particular the filtration of extra virgin olive oil, which employs such a filter medium, has the fundamental characteristic to comprise a succession of steps that consist in:
filling the filter of the machine by filtration with the oil to be filtered,
loading the filter with about 100 kg of filter medium to filter a quantity of olive oil included between 600 and 1300 tons, depending on the origin and quality of the oil (for example the high presence of waxes in the oil increases the amount of filter medium demand especially in winter months), to form the pre-filtration oil cake,
keeping the oil in a substantially constant agitation, until the end of filtration,
keeping the oil at a pressure higher than atmospheric pressure (i.e. “in pressure”), and
ejecting the exhausted panel of filtration. This exhausted panel of filtration is, together with the filtered oil, the final result of the procedure.

Other application examples might include different amounts of filter medium for the same amount of oil to be filtered, nevertheless this is the kind of filter most commonly found in the oil industry.

This exhausted panel consists in exhausted cellulose fiber impregnated with oil, fired from the machine ball.

Advantageously, it may be provided to reintegrate the filter medium to maintain substantially constant the amount of cellulose with respect to the oil to be filtered.

Once the oil has been filtered, it is carried out the unloading of the liquid, normally in an automatic manner, of the filter media and sediments, before washing the filtering meshes in preparation for a subsequent filtration.

The exhausted panel obtained by filtration of virgin olive oil, in particular extra virgin olive oil through the industrial process filtration of extra virgin olive oil as described above, has the main characteristic of being a single byproduct filtration, i.e. a raw material, the second according to the destination.

This exhausted panel, the only product of oil filtration, advantageously includes only oil and cellulose.

The olive oil filtration exhausted panel, in particular for filtration of extra virgin olive oil, can be also used as a supplement in the constitution of the food ration in animal husbandry, in order to obtain a greater palatability for animal feed because it is rich in polyphenols (antioxidants).

Advantageously, the feed thus obtained includes, in addition to the olive oil filtration exhausted panel, also a lipid substance (high quality natural fat).

This lipid substance for example can be constituted by extra virgin olive oil.

The feed thus obtained has a high content of antioxidants, fibers and lipids.

The olive oil filtration exhausted panel obtained by the process of filtration of olive oil, in particular from the filtration of extra virgin olive oil, just described, has molecular characteristics such that they can be used in cosmetics as integral element to obtain cosmetic products and products for cosmetic treatments and which has a concentration of squalene included substantially between 20 and 25% by weight.

In particular, it can be used as an additive in soaps. Necessarily, if the cellulose is not used for the filtration, the exhausted panel is not full of oil and therefore has no squalene necessary for the use in cosmetics

Another use in cosmetics may be that of an active ingredient in cosmetic preparations of various types, such as the treatment of skin blemishes, anti-aging, cell renewal (scrubbing), anti-cellulite, etc. . . .

A third use may be that of component for solar products.

It is also provided the use of said olive oil or extra virgin olive oil filtration exhausted panel as a fuel.

This fuel is preferably subjected to a pellet making process or can be compacted to a socket.

Another use of the olive oil filtration exhausted panel is as a material for composting in industrial plants.

Not least, the filtration exhausted panel can be used as fertilizer. In particular, it may find use as soil conditioner for top soil users mixed with green and quality compost, or as a soil mixed with peat for mushrooming.

From the above description it is thus evident that the invention achieves the intended aim and the intended purposes and in particular it is highlighted the fact that there is provided a filter medium for the filtration of olive oil, in particular for the filtration of extra virgin olive oil, and olive oil filtration process, in particular of the filtration of extra virgin olive oil, which avoids any risk of illness from the operators that are no longer in contact (direct or indirect) with toxic substances such as those contained in the fossil flour.

In particular cellulose is a substance of plant origin and therefore completely free of any danger.

Another advantage of the process according to the invention, is to get in an industrial manner a filtration exhausted panel completely and easily disposable and that, if properly preserved, can be recycled in a totally environmentally friendly in other industrial fields (livestock, cosmetics, fertilization, composting and mushroom farming), giving an advantage for economic development.

Also, the disposal of the traditional exhausted panel (having fossil flour) should be treated by landfill, in addition to direct economic benefits, also brings cost and environmental savings avoiding road transport at the disposal sites.

Another advantage of the filter medium, according to the invention, is given by the fact that the cellulose totally vegetable origin is a raw material practically inexhaustible.

Besides that, the use of only cellulose that goes to totally replace fossil flour instead of producing inert waste to landfills, allows the use of a new byproduct that, in addition to increase the use of vegetable raw material in the cosmetic sector, allows to obtain a raw material for the animal feed rich in lipids and fibers of vegetable origin of high quality which, together, may go to constitute a new preparation, capable of integrating, if not replacing the hay.

In particular in the cosmetics industry, due to the plant origin of the entire humid matrix and due to the type of lipids, antioxidants and vitamins contained in it, it will be extremely positive in terms of validity of the obtainable finished product.

Another advantage of the invention is the fact that, since the cellulose has the ability to retain a greater amount of moisture than the fossil flour, the use of only cellulose for filtration allows to obtain an olive oil more dry that can be stored longer in the bottle.

Further advantages of the exhausted panel obtained from the procedure described are given by the numerous uses to which it may be used as the only byproduct of the oil industry, without the need of other manipulation or chemical and/or mechanical processing.

In particular it can be used as a fuel as it is or in the form of pellet or socket, in composting in industrial plants or even as a fertilizer, as quality soil improver or topsoil.

Another advantageous use of the exhausted panel according to the invention, consists in the fact that from the exhausted cellulosic material can be extracted, by processes known per se, squalene, functional molecule with high commercial value, therefore removing any cost of collection and/or disposal for the oil industry, with obvious improvements on eco-sustainability of the proceedings.

These filter medium, which will completely replace the fossil flour, turn out to be a valid alternative to the use of diatoms which, as mentioned, are running out.

Not last, the use of means readily available in the market and use of common materials, make the device economically competitive.

The invention thus conceived is susceptible of numerous modifications and variations, all falling within the inventive concept.

Moreover, all the details may further be replaced with other technically equivalent elements.

In practice, the materials employed, as well as the dimensions, may be any according to requirements, provided they are consistent with the purpose of embodiment.

1) A filter medium for the filtration of olive oil, in particular for the filtration of extra virgin olive oil, characterized in that it consists of at least 95% by weight, on dry material, of cellulose.

2) The filter medium according to claim 1, characterized by the fact to be constituted by cellulose only.

3) The filter medium according to claim 1, characterized by the fact to comprise a moisture rate included between 5 and 7%.

4) The filter medium according to claim 1, characterized by the fact to comprise a filtering porosity included between 2 and 13 Darcy.

5) An industrial process of filtration of virgin olive oil, in particular the filtration of extra virgin olive oil, characterized by the fact to comprise a succession of steps that consist in:
filling the filter of the machine by filtration with the oil to be filtered,
loading the filter with about 100 kg of filter medium to filter a quantity of olive oil included between 600 and 1300 tons, depending on the origin and quality of the oil (for example the high presence of waxes in the oil increases the amount of filter medium demand especially in winter months), to form the pre-filtration oil cake,
keeping the oil in a substantially constant agitation, until the end of filtration,
keeping the oil at a pressure higher than atmospheric pressure (i.e. “in pressure”), and
ejecting the exhausted panel of filtration, this exhausted panel of filtration is the final result of the procedure, together with the filtered oil.

6) The process according to claim 5, characterized by the fact to reintegrate said filter medium in said filter to maintain substantially constant the amount of cellulose with respect to the oil to be filtered.

7) The process according to claim 5, characterized by the fact to empty said filtered oil, filter media and sediments, before washing filter sectors.

8) An exhausted panel obtained from the filtration of virgin olive oil, in particular extra virgin olive oil, by means of the industrial process of filtration of the extra virgin olive oil according to claim 5, characterized by the fact to be a single byproduct of processing.

9) The exhausted panel according to claim 8, characterized by the fact that said single byproduct comprises only oil and cellulose.

10) An animal feed comprising the olive oil filtration exhausted panel, in particular of extra virgin olive oil, according to claim 8, as a supplement such that the animal feed is rich in polyphenols.

11) The animal feed according to claim 10, further comprising a lipid substance.

12) The animal feed, according to claim 11, characterized by the fact that said lipid substance is constituted by extra virgin olive oil.

13) A cosmetic products and products for cosmetic treatments comprising the olive oil filtration exhausted panel, in particular of extra olive oil filtration, according to claim 8.

14) A fuel comprising the olive oil filtration exhausted panel, in particular of extra olive oil filtration, according to claim 8.

15) The fuel according to claim 14, wherein the fuel has been subjected to a pellet making process.

16) The fuel according to claim 14, wherein the fuel has been compacted to a socket.

17) A material for composting in industrial plants, comprising the olive oil filtration exhausted panel, in particular of extra olive oil filtration, according to one claim 8.

18) A fertilizer comprising the olive oil filtration exhausted panel, in particular of extra olive oil filtration, according to claim 8.
